(19) United States
(12) Patent Application Publication
(10) Pub. No.: US 2024/0374390 A1
(43) Pub. Date: Nov. 14, 2024

(54) SACROILIAC JOINT FUSION IMPLANTS

(71) Applicant: GLOBUS MEDICAL, INC., AUDUBON, PA (US)

(72) Inventor: Matthew Kowalski, Norristown, PA (US)

(21) Appl. No.: 18/315,561

(22) Filed: May 11, 2023

Related U.S. Application Data
(63) Continuation of application No. 18/315,557, filed on May 11, 2023.

(51) Int. Cl. A61F 2/30

(52) U.S. Cl. CPC A61F 2/30988; A61F 2002/30156; A61F 2002/30281; A61F 2002/30405; A61F 2002/30593; A61F 2002/30622; A61F 2002/30858; A61F 2002/30891; A61F 2002/30909; A61F 2002/30919; A61F 2002/30995; A61F 2002/4632

(57) ABSTRACT

Bone implants, assemblies, systems, and methods thereof. The implants and assemblies may be threaded or non-threaded, adjustable or expandable, or otherwise configured to promote fixation and fusion of the sacroiliac joint. The implant may include a screw with a triangular portion along the shaft or a separate triangular cage or sleeve surrounding the screw configured to prevent or minimize rotational motion of the implant.

### CROSS REFERENCE TO RELATED APPLICATIONS

This application is a continuation of U.S. patent application Ser. No. 18/315,557 filed on May 11, 2023, which is incorporated in its entirety herein.

### FIELD OF THE INVENTION

The present disclosure relates to surgical devices, and more particularly, to implants and methods for fusing a sacroiliac joint.

### BACKGROUND OF THE INVENTION

The sacroiliac joint (SI joint or SIJ) is the joint between the sacrum and the ilium bones of the pelvis. Limiting the rotational range of motion (RROM) in the sacroiliac joint has been of increasing interest to surgeons as evidence grows in support of the sacroiliac joint as a pain generator. The sacroiliac joint pain may stem from pregnancy (SIJ dysfunction), adjacent segment disease, trauma, anatomical variations such as excessive lumbar lordosis, osteoarthritic, inflammatory arthritis, etc. In many cases, the etiology may be treated with fusion of the sacroiliac joint.

Development in implants aim to ensure fixation and promote rapid fusion. Patients that receive sacroiliac joint fixation receive revision surgery 30.8% of the time compared to only 5.7% of patients that receive sacroiliac joint fusion. The revisions may be due to loosening of the screws, which lead to the reoccurrence of sacroiliac joint pain. As such, there currently exists a need to combine the clinical benefit of limiting the rotational range of motion, while being able to provide fixation and precise insertion methods of an implant to provide fusion.

### SUMMARY OF THE INVENTION

To meet this and other needs, implants, assemblies, systems, and methods are provided. In particular, the sacroiliac joint may be fixated and/or fused via a threaded or non-threaded implant that may prophylactically fuse the sacroiliac joint. The implant may include a screw with a triangular portion along the shaft or a separate triangular cage or sleeve surrounding the screw configured to prevent or minimize rotational motion of the implant. The implant may be adjustable, expandable, or otherwise configured to promote fixation and fusion of the sacroiliac joint. These implants may be used in bilateral, open, and percutaneous approaches to the spine and/or ilium and may be compatible with robotic and/or navigation systems.

According to one embodiment, a sacroiliac implant includes a shaft extending along a central longitudinal axis having a distal tip configured to facilitate insertion into bone and a proximal end configured to be engaged by an instrument. The shaft defines an external thread configured to enhance insertion and/or engagement with bone. A portion of the shaft includes a polygonal portion with a triangular cross-section having three faces. The polygonal portion is configured to limit rotational motion about the central longitudinal axis of the shaft.

The sacroiliac implant may include one or more of the following features. The polygonal portion may be an equilateral triangle with three equal faces. The three faces may define three vertices at each corner. The three faces may be planar. The polygonal portion may be a core of the shaft. The polygonal portion may define a lattice structure. The thread may be a helical thread. The implant may include a graft window extending longitudinally through the shaft.

According to one embodiment, a sacroiliac implant includes an inner core having a triangular cross-sectional profile and a circumscribed helical thread circling the inner core. The triangular cross-sectional profile includes three planar faces joined at three vertices, which are truncated. The inner core supports the helical thread such that the thread contacts and is supported by the truncated vertices. The thread does not contact the planar faces such that gaps are created between the thread and the inner core, thereby allowing for boney in-growth in the gaps.

According to one embodiment, a sacroiliac implant includes an implant having a triangular shaft with three flattened surfaces and three rounded vertices, and one or more threads or flutes following an outer profile of the triangular shaft. The triangular shaft is configured to provide rotational and axial fixation in the bone.

According to one embodiment, a sacroiliac implant includes an outer triangular cage having three faces encapsulating an inner driving screw. A longitudinal windows in each face of the cage permits the inner driving screw to project therefrom. The triangular cage is free to rotate relative to the driving screw and is configured to prevent rotation of the implant or movement of the joint once implanted.

According to one embodiment, a sacroiliac implant includes a shank having a triangular shape with continuous reliefs coaxial to the length of the screw separating alternating threaded portions. Each relief terminates into an adjacent threaded portion with a sharpened cutting edge on one side of the relief such that the implant is configured to rotate in one direction to cut and self-harvest bone.

According to one embodiment, a sacroiliac implant includes a central screw with a plurality of barbs forming a triangular profile in order to displace shear and torsional forces through the screw. The central screw defines a plurality of channels co-axial to the length of the screw configured to receive the respective barbs. Each of the barbs includes a tongue slidable into the respective channel. The central screw may define an internally threaded cannulation configured to pull the barbs along the respective channels.

According to one embodiment, a sacroiliac implant includes an outer cage and an internal screw aligned along a central longitudinal axis. The outer cage has a polygonal outer shape configured to limit rotational motion. The internal screw is sized and dimensioned to fit through the outer cage. The internal screw has an enlarged head and a shaft with one or more threaded sections. The outer cage is free to rotate relative to the internal screw.

The sacroiliac implant may include one or more of the following features. The outer cage may have a triangular cross-section. The outer cage may include three faces and three vertices. The shaft of the internal screw may include one or more narrowed shaft portions, and the outer cage may define corresponding narrowed sections configured to mate with the narrowed shaft portions such that the narrowed shaft portions act as stops to prevent the outer cage from translating along the central longitudinal axis. The narrowed shaft portions may be non-threaded and have a reduced diameter relative to the threaded sections. The shaft may include three distinct sections including a distal section with a tapered nose, a middle section having a triangular profile, and a rear section having a cylindrical profile. The outer cage may include one or more prongs extending distally from the cage. The implant may include a spiral sweep, for example having a lattice structure, interrupting the threaded sections.

According to one embodiment, a method for stabilizing a sacroiliac joint includes (a) providing an implant having an outer cage with a polygonal cross-section and an internal screw aligned along a central longitudinal axis, wherein the outer cage is free to rotate about the central longitudinal axis relative to the internal screw to allow for minimal displacement of patient tissue; (b) accessing an ilium of a patient; and (c) inserting the implant across the sacroiliac joint such that once the implant is fully seated, the outer cage acts as a stop to prevent rotational motion of the implant. The method may include inserting multiple implants across the sacroiliac joint to better stabilize and prevent movement of the joint. The method may include accessing the sacroiliac joint with a robotic and navigational system.

According to one embodiment, a sacroiliac implant includes a screw shaft, a triangular cage, and a washer, configured to provide compression across the sacroiliac joint. The triangular cage includes a head and body threadedly coupled to the screw shaft. The washer includes a proximal flexible portion for engaging the head of the triangular cage and a distal bone engaging portion to engage bone. The washer is configured to angulate with respect to the triangular cage and screw shaft, thereby enabling the washer to conform to various angles of the bone while the implant is advanced across the sacroiliac joint.

According to one embodiment, a sacroiliac implant includes an expandable implant with a front section, a first expandable section, a second expandable section, and a rear section. An inner shaft pulls the front section toward the rear section and the expandable sections move against ramps on the respective sections, thereby expanding the expandable sections radially outward. The expandable implant provides compression with additional radial pressure on the bone by translating the compressive force radially.

According to one embodiment, a sacroiliac implant includes an expandable implant with a central screw and an expandable outer sleeve. The outer sleeve includes a cylindrical tube having one or more expandable bars. The sleeve or portions thereof may be configured to radially expand via cement or cement filled balloons, for example.

According to one embodiment, a sacroiliac implant includes an inner shaft receivable through an outer shaft. The inner and outer shafts include helical cuts or graft windows defined through the walls of both shafts. Rotational movement of inner and/or outer shafts create a movable lumen across the body of the implant. The final position of the shafts may be stopped using a one-way ratchet lock. In yet another embodiment, the position between the shafts is determined by interlocking splines.

Also provided are kits including implants of varying types and sizes, bone fasteners, spinal rods, k-wires, insertion tools, instruments, bone cement, biomaterials, and other components for performing the procedure(s).

### BRIEF DESCRIPTION OF THE DRAWINGS

A more complete understanding of the present invention, and the attendant advantages and features thereof, will be more readily understood by reference to the following detailed description when considered in conjunction with the accompanying drawings, wherein:

FIGS. 1A-1C show a triangular core screw with circumscribed helical threads circling the triangular core according to one embodiment;

FIGS. 2A-2C show a triangular implant with exterior threads having three flat surfaces on a sacroiliac joint screw according to one embodiment;

FIGS. 3A-3C show a triangular implant having a triangular outer cage encapsulating a driving screw according to one embodiment;

FIGS. 4A-4B show a triangular implant with continuous reliefs coaxial to the length of the screw separating alternating threaded portions according to one embodiment;

FIGS. 5A-5E show a triangular implant with barbs and a central drive screw according to one embodiment;

FIGS. 6A-6D show a triangular implant with fluted cutting features according to one embodiment;

FIGS. 7A-7B show an expansion anchor screw with a threaded screw shank, expansion tube, slip ring, and threaded compression nut according to one embodiment;

FIGS. 8A-8B show a triangular screw with a taper from a circular cross-section to a triangular cross-section according to one embodiment;

FIGS. 9A-9C show a triangular screw where the proximal end starts with a circular profile and transitions to a triangular profile according to one embodiment;

FIGS. 10A-10B show a triangular screw where the profile changes from an inscribed circle to a triangular profile according to one embodiment;

FIGS. 11A-11D show a central screw with a freely rotatable triangular cage according to one embodiment;

FIG. 12 shows a central screw with an outer cage according to one embodiment;

FIGS. 13A-13D show a screw with a profile transitioning from a circumscribed circle to a triangle and including lattice portions for boney ingrowth according to one embodiment;

FIGS. 14A-14G show a central screw having lattice portions with a triangular outer cage according to one embodiment;

FIGS. 15A-15D show a sacroiliac joint system with a triangular cage around the central screw and an angulating washer according to one embodiment;

FIGS. 16A-16C show an expanding pin implantable screw according to one embodiment;

FIGS. 17A-17B show an expanding screw with a sleeve having expandable bars according to one embodiment;

FIGS. 18A-18D show an implant with inner and outer shafts with movable helical cuts according to one embodiment; and

FIG. 19 shows an implant with a splined inner shaft and outer shaft with moveable graft windows or lumens according to one embodiment.

### DETAILED DESCRIPTION OF THE INVENTION

Implants, assemblies, and systems are configured to fixate and/or fuse the sacroiliac joint. The implants may be threaded or non-threaded, adjustable or expandable, or otherwise configured to promote bone fixation and/or prophylactically fuse the sacroiliac joint. In some embodiments, the implant may incorporate a titanium triangular implant feature or component configured to effectively limit the rotational range of motion, while providing fixation and promoting fusion. The bone implants may be used independently or may include the capability to integrate with long rod constructs, for example, with a tulip or other suitable attachment interface, to prophylactically fuse the sacroiliac joint.

These implants may be used in bilateral, open, and percutaneous approaches to the spine and/or ilium and may be compatible with robotic, imaging, and/or navigation systems. Further details of robotic and/or navigational systems can be found, for example, in U.S. Pat. Nos. 10,675,094, 9,782,229, and U.S. Patent Publication No. 2017/0239007, which are incorporated herein by reference in their entireties for all purposes.

Although described herein with reference to the sacroiliac joint, it will be appreciated that the devices described herein may be applied to other areas of the spine, other orthopedic locations in the body, and other medical procedures, such as trauma applications. Any of the implants described herein may be offered in a multitude of styles, diameters, and lengths, helping to ensure optimal patient fit.

The implants or components thereof may be comprised of titanium, stainless steel, cobalt chrome, cobalt-chrome-molybdenum, tungsten carbide, carbon composite, plastic or polymer—such as polyetheretherketone (PEEK), polyethylene, ultra-high molecular weight polyethylene (UHMWPE), resorbable polylactic acid (PLA), polyglycolic acid (PGA), allograft, autograft, or combinations of such materials or any other appropriate material that has sufficient strength to be secured to and hold bone, while also having sufficient biocompatibility to be implanted into a body. Although the above list of materials includes many typical materials out of which implants may be made, it should be understood that implants comprised of any appropriate material are contemplated.

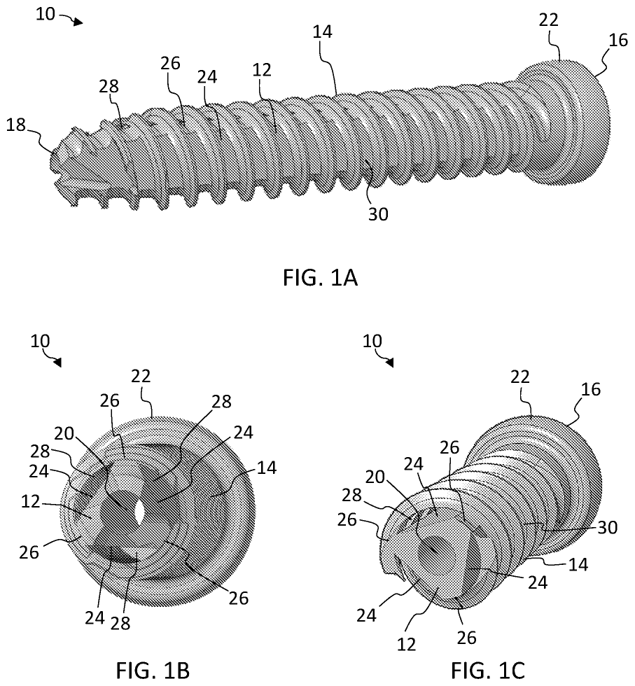

Turning now to the figures, where like reference numbers may refer to like elements, FIGS. 1A-1C shows an orthopedic fixation device, bone fastener, or triangular implant 10 according to one embodiment. The implant or bone fastener 10 may include a triangular core 12 and one or more helical threads 14 configured to engage bone. The helical threads 14 may be further configured to allow maximum harvesting of boney material during insertion. The implant 10 extends from a proximal end 16 to a distal end 18 along a central longitudinal axis. The implant 10 defines a central cannulation 20 along the central longitudinal axis between the proximal and distal ends 16, 18. The central cannulation 20 may include a generally cylindrical bore extending through the body of the implant 10.

The proximal end 16 may include an enlarged head 22. The head 22 may be partially or fully rounded, cylindrical, spherical, or otherwise configured to engage bone and/or interface with a modular tulip element or head (e.g., for securing a spinal rod). The outer perimeter of the head 22 may be smooth (as shown), threaded, or provided with a roughened or textured surface. The proximal portion of the head 22 may define one or more drive and/or engagement surfaces, such as a hexalobular socket, for example, that can be engaged by a driving instrument or other instrument. It will be appreciated that any suitably shaped tool engagement drive recess may be provided to insert the implant 10 into bone.

The implant includes an elongated shank or inner core 12 connected to the head 22. The core 12 terminates at a distal tip at the distal end 18 of the implant 10, which may be blunt, pointed, or otherwise configured to engage bone. As best seen in the partial cross-section shown in FIG. 1C, the inner core 12 may define a polygonal cross-section, such as a triangular cross-section. The triangular core 12 may include three surfaces or faces 24 and three corners or vertices 26. The surfaces or faces 24 may be generally flat or planar faces. The vertices 26 may be truncated creating a facet in place of each vertex to engage the helical threads 14. In one embodiment, the triangular core 12 is an equilateral triangle with three equal planar faces 24. The triangular core 12 is configured to provide rotational and axial fixation in the bone.

The triangular implant 10 includes one or more circumscribed helical threads 14 circling the triangular core 12. The threaded portion 14 may function as a helical retaining interface with the bone. The external threads 14 may include a single lead, dual lead, or multi-lead thread. In one embodiment, the helical threads 14 includes a double helix to allow for screw-like insertion. The threaded portion 14 allows for easy insertion and incorporation of navigation. The defined length of implant 10 may allow for integration with navigation and robotic platforms.

The triangular core 12 supporting helical threads 14 allow for screw-like insertion, fixation, promotion of rapid on-growth and fusion, and capture of displaced boney material in the internal sections. The external threads 14 may extend along the length of the triangular core 12 or a portion thereof, such that the threads 14 contact and are supported by each of the truncated vertices 26 of the core 12. The threads 14 do not contact the planar faces 24, thereby creating gaps or channels 28 between the thread portions 14 and the triangular core 12. The channels 28 are configured to capture displaced boney material therein. The boney material is trapped in the channels 28 created by the space between the helix 14 and the core 12. These gaps or channels 28 may be in fluid communication with the open area 30 between each adjacent thread portion 14 to guide the bone material into the inner sections 28. One or more graft windows may also be included to provide maximum fusion. Rapid on-growth may occur when the helix 14 acts as a net by capturing boney material created by moving the triangular core 12 in a screw-like manner. The fixation created by fusion may be increased because the bone is able to grow around the helical sweeps 14 faster than a bone bridge formed through a lumen that spans the entirety of a typical screw diameter.

Implant 10 has the benefits of no impaction and may be integrated with robotics and navigation systems. Implant 10 may be 3D printed to enhance boney on-growth potential. Implant 10 may be coated and may have graft windows along the inner core 12 to further promote boney on-growth. In one embodiment, implant 10 may include a lag screw feature with a back fillable feature. The lag option may provide added fixation through compression of the joint.

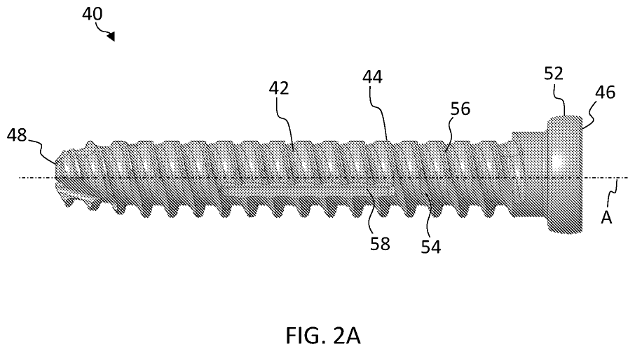

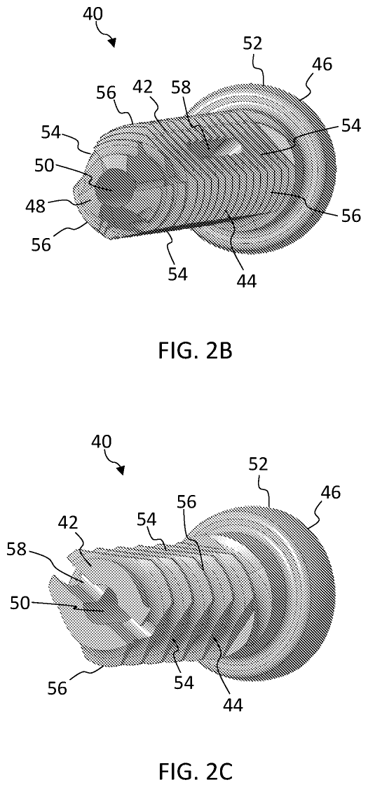

Turning now to FIGS. 2A-2C, an orthopedic fixation device, bone fastener, or triangular implant 40 is shown according to one embodiment. In this embodiment, three flat surfaces 54 are provide on the sacroiliac joint screw to limit rotational motion and promote fusion. The implant or bone fastener 40 may include a shaft 42 and one or more threads 44 configured to engage bone. The implant 40 extends from a proximal end 46 to a distal end 48 along a central longitudinal axis A. The implant 40 defines a central cannulation 50 along the central longitudinal axis between the proximal and distal ends 46, 48. The central cannulation 50 may include a generally cylindrical bore extending through the body of the implant 40.

The proximal end 46 of implant 40 may include an enlarged head 52, similar to head 22, and an elongated shank or shaft 42 connected to the head 52. The shaft 42 includes one or more threads 44. The screw thread 44 may include an external thread with a helical ridge defined by a helical groove wrapped around the shaft 42. The thread 44 may extend the entire length or a partial distance along the length of the shaft 42. Each screw thread section may include a crest at the top-most point or outer-most surface of the ridge and a root at the bottom-most point or inner-most surface of the groove. The external threads 44 may include a single lead, dual lead, or multi-lead thread configured to allow for screw-like insertion into bone.

As best seen in FIGS. 2B-2C, the threaded shaft 42 includes one or more flattened sections or surfaces 54. In particular, the threads 44 may be truncated to form flat or planar sections of thread. In this manner, the outer perimeter of the threaded shaft 42 may define a generally polygonal cross-section, such as a triangular cross-section. The triangular threaded shaft 42 may include three flat surfaces 54 and three rounded vertices 56. In one embodiment, the triangular threaded shaft 42 approximates an equilateral triangle with three equal planar surfaces 54. The triangular shape of the exterior threads 44 may be configured to provide rotational and axial fixation in the bone.

In one embodiment, the implant 40 includes one or more graft windows 58 in fluid communication with the central cannulation 50. The graft window 58 may extend along a length of the shaft 42. Implant 40 may be machined or 3D printed. The implant 40 may include various arrangements of multiple graft windows, surface treatments, or other features to enhance fusion. Other embodiments may also include a lag screw option creating compression between the joints for added fixation before fusion. The benefits of implant 40 include a simplified design with good fixation that maximizes the strength by maximizing material. The triangular outer shape of the sacroiliac joint screw is configured to limit rotational motion and promote fusion.

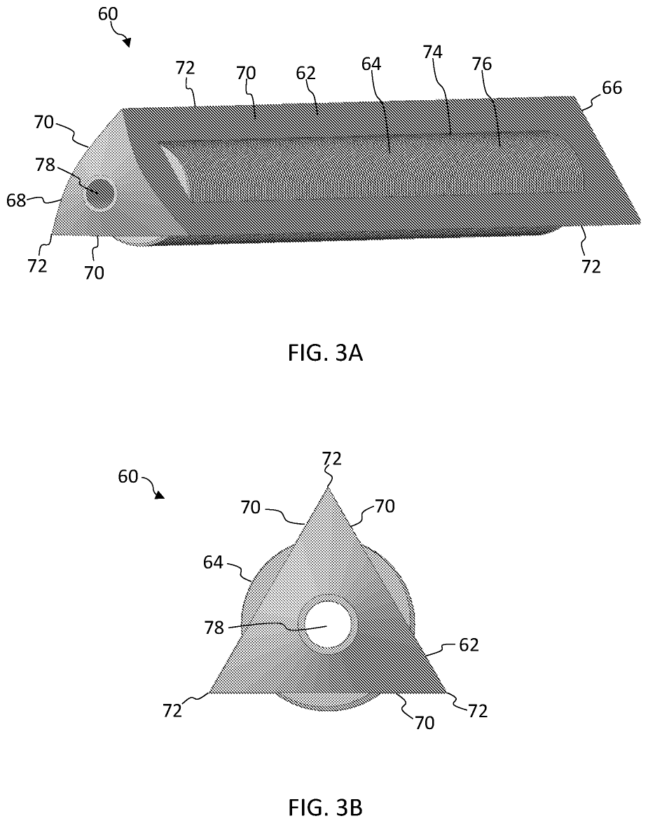

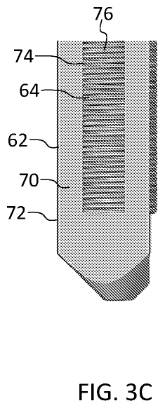

Turning now to FIGS. 3A-3C, an orthopedic fixation device, bone fastener assembly, or triangular implant 60 is shown according to one embodiment. In this embodiment, the implant 60 includes an outer triangular cage 62 encapsulating an inner driving screw 64. The implant 60 extends from a proximal end 66 to a distal end 68 along a central longitudinal axis. Instead of a rigidly fixed triangular dowel or prism screw, the triangular cage 62 is free to move relative to the driving screw 64.

Implant 60 includes outer cage 62, which may have a polygonal cross-section, such as a generally triangular cross-section. The triangular cage 62 may include three sides or faces 70 and three corners or vertices 72. The sides 70 may be planar or curvilinear forming sharpened corners or vertices 72. The faces 70 of the cage 62 may be smooth or textured. A surface texture, such as laser engraved surface texture, titanium plasma spray (TPS) coating, or 3D texturing may be added to promote bony on-growth. The proximal and distal ends 66, 68 may also be planar or curvilinear. In one embodiment, the one or both of the ends 66, 68 are convexly curved.

The cage 62 is hollow and retains screw 64 therein. The screw 64 is captured completely by the body of the cage 62. One or more longitudinal windows 74 may be provided in the faces 70 of the cage 62 allowing the internal screw 64 to project therefrom. As best seen in FIG. 3B, each face 70 may include a longitudinal window 74 to allow the internal screw 64 to protrude past the sides 70 of the outer cage 62. Once implanted, the triangular shape of the cage 62 may help to prevent rotation of the implant or movement of the joint.

The internal screw 64 includes a shank with one or more threads 76. It will be appreciated that the threads 76 may have a number of different features, such as lead(s), thread pitch, thread angle, shaft diameter to thread diameter, overall shaft shape, and the like configured to engage bone. The implant 60 may include a central cannulation 78 along the central longitudinal axis. The central cannulation 78 may extend through the entire screw body 64 and through the proximal and distal ends 66, 68 of the cage 62.

The internal screw 64 may be rotated to drive the triangular cage 62 through the bone and into a final location. The triangular cage 62 is free to move relative to the driving screw 64. This is due to the fact that the cage 62 is able to float on the screw 64 when inserted and move coaxially with the driving screw 64. Once implanted, however, the cage 62 is not able to rotate as it gets purchase into the bone space. During insertion, the bone provides the counter rotational barrier, but once fully inserted, the triangular cage 64 prevents the implant from rotating.

The outer cage 64 is independent and uncoupled from the inner drive 64. When the implant 10 engages the bone, it will drive into the bone and push the triangular cage 62. The bone itself prevents the triangle cage 62 from spinning. Once inside the body, the screw 64 becomes a structural support for the triangular cage 62. In one embodiment, the outer cage 62 is not coupled to the screw drive 64, and does not connect to a longer construct, to allow a multiplicity of approaches.

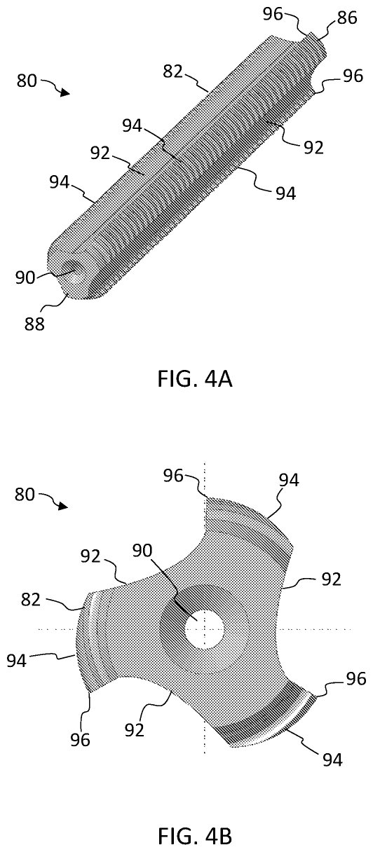

Turning now to FIGS. 4A-4B, an orthopedic fixation device, bone fastener, or triangular implant 80 is shown according to one embodiment. In this embodiment, the implant 80 includes a one-sided cutting edge 96 configured for self-harvesting bone. The implant 80 includes a shank 82 extending from a proximal end 86 to a distal end 88 along a central longitudinal axis. The shank 82 defines a central cannulation 90 therethrough, such as cylindrical bore, extending along the central longitudinal axis between the proximal and distal ends 86, 88.

The shank 82 may have a generally triangular outer shape with a plurality of reliefs 92 separating threaded portions 94. The reliefs 92 may include a continuous recess or groove extending between the proximal and distal ends 86, 88 of the shank 82. As best seen in FIG. 4B, in the axial direction, the reliefs 92 may be asymmetrical. The reliefs 92 may include three reliefs 92 separated by three alternating threaded sections or portions 94. Although three reliefs 92 are exemplified in shank 82, it will be appreciated that any suitable number and type of relief may be selected. The threaded areas 94 may include one or more external threads, such as helical threads about the face of the shank 82. The threads may have any suitable angle, lead, pitch, etc. to enhance insertion and/or engagement with the bone.

Each relief 92 may terminate into the adjacent threaded portion 94 with a sharpened or pointed cutting face or edge 96. The cutting edge 96 may be provided on one side of the relief 92 such that the implant 80 is configured to rotate in one direction to cut and self-harvest bone. The cutting edge 96 does not overhang but does cut pathways for the pitch of the helical sweeps of the threaded portions 94. The relief 92 preceding the cutting face 96 allows for easy back out and repositioning, for example, when not using navigation and robotics. By rotating the implant 80 into its final position, the cutting edges 96 maximize the amount of bone to fill the void and provide faster fusion.

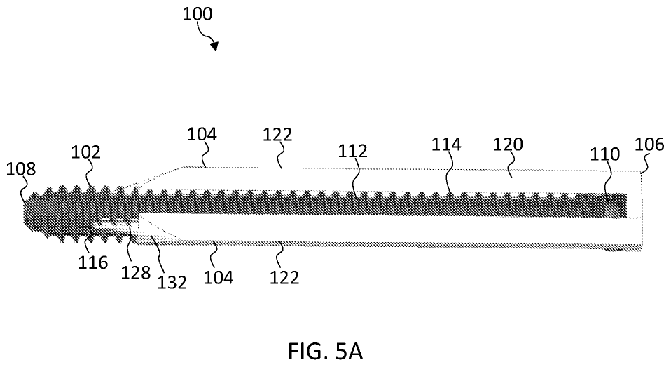

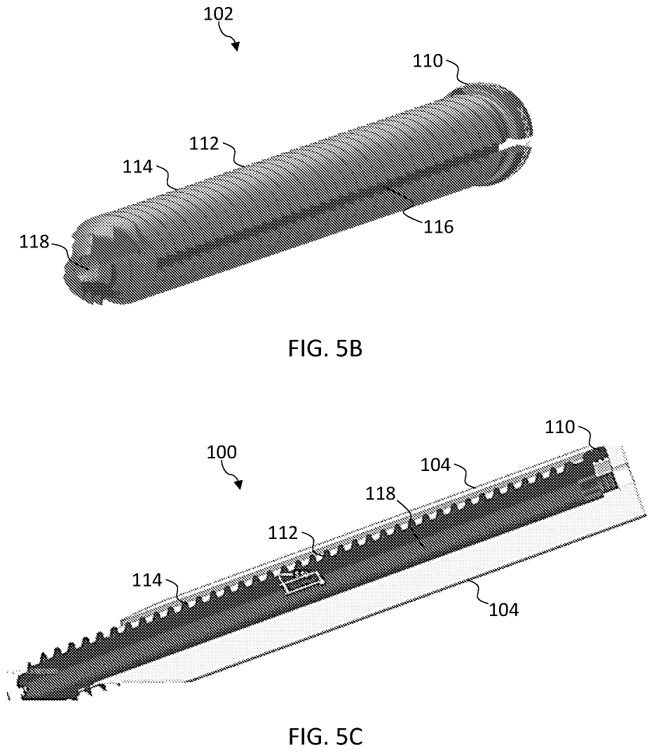

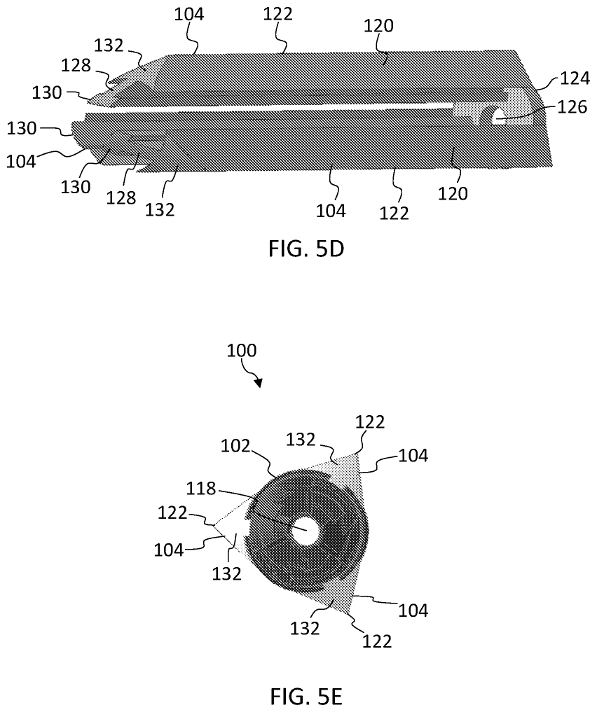

Turning now to FIGS. 5A-5E, an orthopedic fixation device, bone fastener assembly, or triangular implant 100 is shown according to one embodiment. In this embodiment, the implant 100 includes a central screw 102 and a plurality of barbs 104 forming a triangular profile in order to displace shear and torsional forces through the screw 102. The implant 100 extends from a proximal end 106 to a distal end 108 along a central longitudinal axis.

As best seen in FIG. 5B, the main body or central screw 102 may be similar to a sacroiliac joint screw with an enlarged head 110 and an elongated shaft 112 connected to the head 110. The shaft 112 includes one or more external threads 114, for example, with a helical ridge defined by a helical groove wrapped around the shaft 112. The thread 114 may extend the entire length or a partial distance along the length of the shaft 112. The external threads 112 may include a single lead, dual lead, or multi-lead thread configured to allow for screw-like insertion into bone. The main screw body 102 provides assured robotic placement and provides strength to the entire construct. The implant 100 may have optional graft windows and may have a lag screw main body option.

In this embodiment, the sacroiliac joint screw 102 is modified with one or more channels 116 configured to receive and guide the respective barbs 104. Each channel 116 is a straight longitudinal channel coaxial to the length of the screw 102. Each longitudinal channel 116 extends from the proximal end of the screw 102 along a distance of the shaft 112 stopping short of the distal end of the screw 102. The channels 116 provide a rigid clear pathway for the barbs 104 to travel along the length of the screw 102. For example, the screw 102 may include three equally spaced channels 116 that provide pathways for each of the barbs 104 to be pulled in. The screw 102 may define a central cannulation 118 therethrough. The central cannulation 118 defines an internal thread configured to provide a mechanical advantage to pull the barbs 104 along the channels 116. The internal thread 118 of the screw 102 allow the barbs 104 to be inserted with ease and precision, and allow for barb removal, if necessary, for easy revision.

With further emphasis on FIG. 5D, each barb 104 includes an elongate longitudinal body 120 that spans along the length of the screw shank 112. Each barb 104 includes a pointed or sharpened outer-facing edge 122 configured to engage bone. When three barbs 104 are arranged about center screw 102, the barbs 104 form a generally triangular shape or profile. As best seen in FIG. 5E, each outer edges 122 generally form the vertices of the outer triangular shape. The triangular profile of barbs 104 may help to minimize the displacement of bony material in the patient. The proximal end of each barb 104 may be affixed to a base 124 to maintain the arrangement of barbs 104. A central bore 126 may be defined through base 124, which coaxially aligns with central cannulation 118 through screw 102.

Each barb 104 defines a tongue 128 configured to be received in the respective channels 116 in the screw 102. The tongue 128 may define an inner projection with an enlarged tip 130 at its free end. The tongue 128 and enlarged free end 130 may have a generally T-shaped cross-section. In this manner, the tongue 128 and channel 116 may form a tongue and groove, dovetail, or other suitable mating structure configured to guide and secure the barbs 104 to the screw 102. The tongue 128 may extend along the entire length of the barb body 120. The tongue portion 128 may project distally past the end of barb body 120 and a distal portion of the barb body 120 may have an angled or tapered face 132 leading into sharpened edge 122. The barbs 104 may have a contoured bottom face to match the screw outer diameter to provide additional support of the construct in shear. Implant 100 minimizes the radial distance of the barbs 104 to the main body 102 while increasing the surface area contact in order to displace shear and torsional forces through the main body.

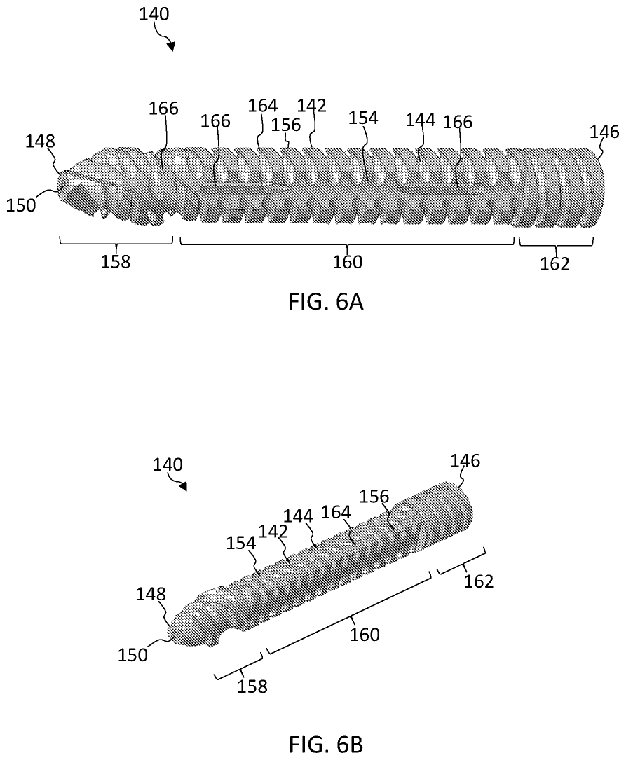

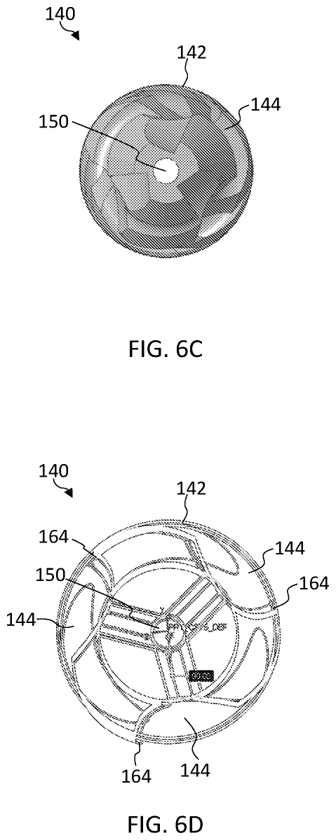

Turning now to FIGS. 6A-6D, an orthopedic fixation device, bone fastener, or triangular implant 140 is shown according to one embodiment. Implant 140, similar to implant 40, includes one or more flat surfaces 154 configured to limit rotational motion and promote fusion. In this embodiment, the threads may be replaced by a series of flutes 144 to cut and self-harvest bone to provide faster fusion. The implant 140 may include a shaft 142 extending from a proximal end 146 to a distal end 148 along a central longitudinal axis. The implant 140 may define a central cannulation 150 therethrough.

In this embodiment, the implant 140 includes a flute system 144 creating a triangular profile and a different helical profile of the retaining flutes 144. The flutes 144 start with a modest pitch, increase in pitch, and then transition to a straight shank 142. A distal section 158 includes a tapered nose or distal tip and the start or lead in of flutes 144. A middle section 160 may include a triangular profile with three planar or flat surfaces 154 and three rounded vertices 156. The flutes 144 in section 160 may each include curvilinear grooves or recesses defining a cutting edge 164. The cutting edge 164 may be distal-facing to promote self-harvesting during insertion. A rear section 162 may include a cylindrical portion with a helical sweep for flutes 144. The retaining flutes 144 may have a circular profile with chamfered ends, which may allow bone to be captured. This configuration may also hold two boney segments together by traversing the segments or in parallel with the segment surfaces. This profile is configured to move boney material to the area around the middle section 160 of shank 142. Implant 140 may be formed via additive manufacturing (e.g., 3D printing) or traditional machining. Implant 140 may also include a lag screw design and one or more optional graft windows 166. The triangular outer shape of the sacroiliac joint screw 140 is configured to limit rotational motion and promote fusion.

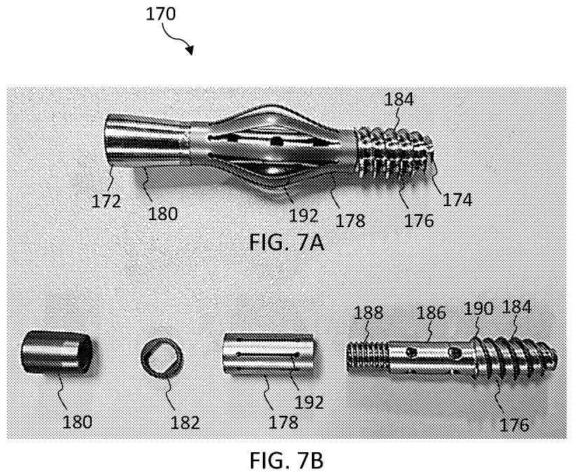

Turning now to FIGS. 7A-7B, an expansion anchor screw or expandable bone anchor implant 170 is shown according to one embodiment. The expandable bone anchor 170 has a collapsed configuration, thereby allowing the anchor 170 to be inserted into bone, for example, in a minimally invasive manner. Once inserted in the sacroiliac joint, the bone anchor 170 may be expanded into an expanded configuration, as best seen in FIG. 7A, thereby creating a strong press-fit in the bone.

The expandable anchor 170 extends from a first proximal end 172 to a second distal end 174 along a central longitudinal axis. As best seen in the exploded view of FIG. 7B, the expandable anchor 170 includes a screw body 176, an expandable collar 178, a threaded cap 180, and a slip ring 182. The screw body 176 includes a threaded distal section 184, a central section 186, and a threaded proximal section 188. The distal end 184 may have a distal tip that is blunt, pointed, or otherwise configured to engage bone. The threaded distal section 184 may include one or more threads with a suitable angle, lead, pitch, etc. to enhance insertion and/or engagement with the bone. The central section 186 may have a cylindrical body configured to receive the expandable collar 180. One or more openings may be provided into the central section 186. A lip 190 may separate the central section 186 from the distal section 184. The threaded proximal section 188 includes one or more threads configured to mate with internal threads in the threaded cap 180. The threaded proximal section 188 may define a recess configured to receive an instrument for inserting the implant 170.

The collar 178 may be a hollow tube with a generally cylindrical outer shape in its collapsed configuration (shown in FIG. 7B). The collar 178 is receivable over the central section 186 of the screw 176. The collar 178 defines a plurality of longitudinal slits 192, for example, equally spaced around the center of the collar 178. When in its initial collapsed configuration, the slits 192 may be arranged in parallel about the body of the collar 178. When the collar 178 is compressed, the beams between the slits 192 buckle or bend outwardly, thereby forming the expanded position, via flexion-based expansion (shown in FIG. 7A). It will be appreciated that various profile geometries and slit configurations may be utilized in order to expand the collar 178.

In an alternative embodiment, the expandable collar 178 has a generally triangular cross-section. The expanded collar 178 is configured to actively fill the void opened from the threaded screw head 176 and the inscribed triangle. A triangular shape for collar 178 may help to increase fusion rates as the bone may grow around the expanded sections before fusion through the graft windows. With a lag functionality, the threaded compression nut 180 may be threaded to create compression in the joint before compression of the expanded triangular collar 178 to increase fixation before fusion.

The anchor 170 may be inserted and then expanded around the middle by rotation of the threaded cap 180. The threaded cap 180 is cannulated with a threaded interior configured to engage with the threaded proximal section 188 of the screw 176. As the threaded cap 180 is rotated, an axial force is applied to the collar 178. The collar 178 is squeezed between the threaded cap 180 and the lip 190 on the screw 176, thereby causing a deformation of the collar 178. The slip ring 182 may be a circular ring positionable between the threaded cap 180 and the collar 178. The circular slip ring 182 may help to reduce friction in the mechanism during expansion.

One or more implants 170 may be inserted using a lateral or posterior approach to the sacroiliac joint, for example. A traditional driver or other suitable instrument may install the anchor 170 and then the central collar 178 may be expanded outwardly. The expansion mechanism works without hinges or other moving parts, as may be visible in a threaded ramp based mechanism. Some benefits of a flexion-based expansion mechanism are simple parts and a fewer part count.

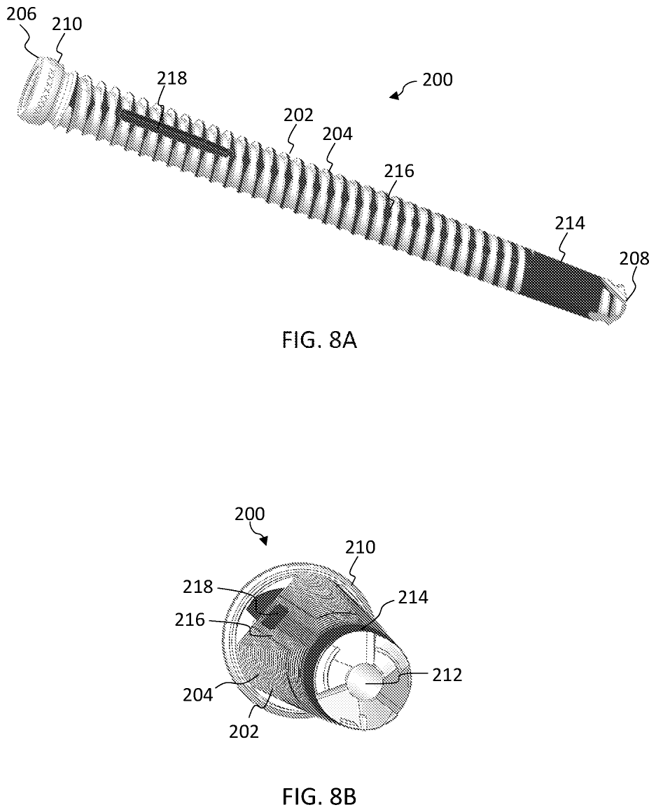

Turning now to FIGS. 8A-8B, an orthopedic fixation device, bone fastener, or triangular implant 200 is shown according to one embodiment. Implant 200 includes one or more flat portions 216 configured to limit rotational motion and promote fusion. In this embodiment, the implant 200 tapers from a circular cross-section to a triangular cross-section. The implant 200 may include a shaft 202 extending from a proximal end 206 to a distal end 208 along a central longitudinal axis. The proximal end 206 includes an enlarged head 210. The implant 200 may define a central cannulation 212 therethrough.

The shaft 202 defines a threaded portion 204, which may include one or more threads with a suitable angle, lead, pitch, etc. to enhance insertion and/or engagement with the bone. In this embodiment, the thread profile remains perpendicular from the head face of the implant 200. A distal portion 214 of shaft 202 may be non-threaded. The non-threaded portion 214 may have a generally circular cross-section. The shaft geometry may transition along its length from the circular cross-section to a non-circular cross-section. For example, the shaft 202 may include one or more flat or planar sections 216 or a portion of the thread 204 may be truncated to form a flat or planar area 216. In one embodiment, the shaft 202 may include three flat or planar sections 216, thereby forming a generally triangular cross-sectional shape. The implant 200 may taper from the circular cross-section near the distal end 208 to the triangular cross-section toward the proximal end 206. The implant 200 may also include one or more graft windows 218 to further facilitate fusion. Implant 200 is configured to insert like a screw and gain more purchase as it is inserted. The profile transitions into a triangle for rotational stability when fused.

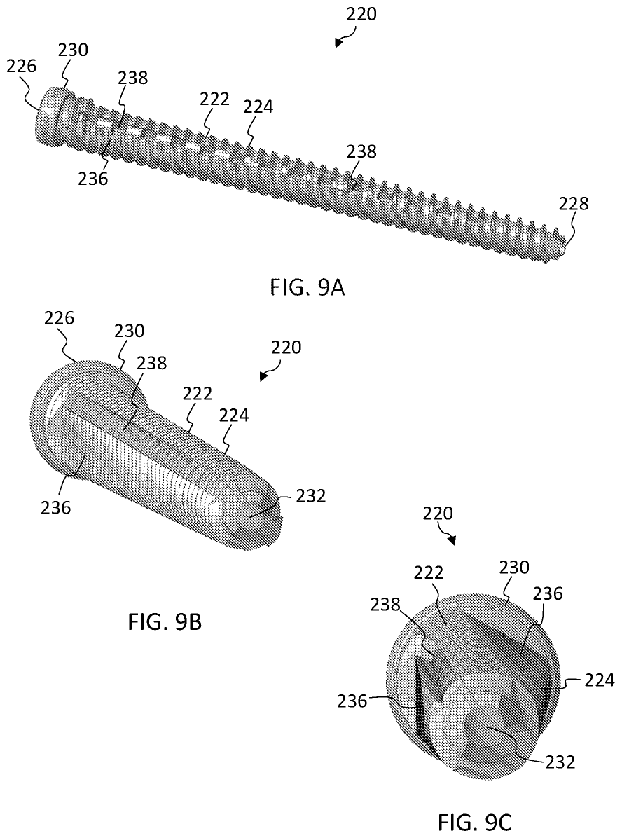

Turning now to FIGS. 9A-9C, an orthopedic fixation device, bone fastener, or triangular implant 220 is shown according to one embodiment. Implant 220, similar to implant 200, transitions from a circular cross-section to a triangular cross-section, which includes flat portions 236 configured to limit rotational motion and promote fusion. The implant 220 may include a shaft 222 extending from a proximal end 226, with an enlarged head 230, to a distal end 228 along a central longitudinal axis. The implant 220 may define a central cannulation 232 therethrough.

The shaft 222 may be threaded 204 along its length. The external threads 204 may include any suitable angle, lead, pitch, etc. to enhance insertion and/or engagement with the bone. The shaft geometry may transition along its length from a circular cross-section to a non-circular cross-section. For example, the shaft 222 may include one or more flat or planar sections 236, such as three flat sections 236, thereby forming a generally triangular cross-sectional shape. In one embodiment, one end 228 starts with a circular profile and transitions to a triangular profile as it moves closer to the head 230. The thread profile follows the transition from the inner screw blank from inscribed circle to triangular, so the thread depth and pitch are consistent across the length of the screw 220. The implant 220 may have optional graft windows 238 and a self-harvesting channel to facilitate fusion.

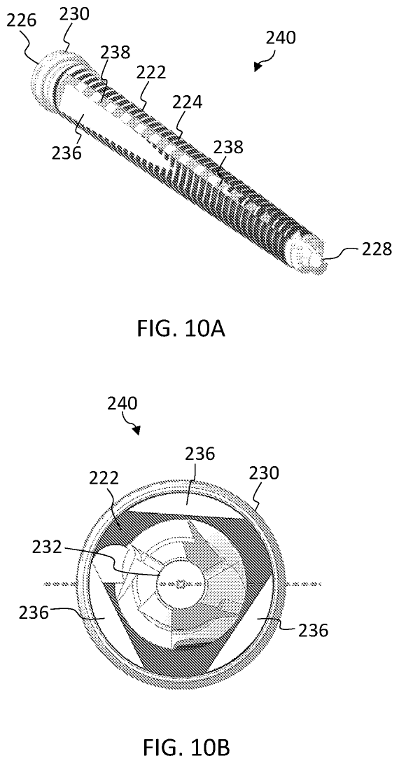

Turning now to FIGS. 10A-10B, triangular implant 240 is similar to triangular implant 220, where the profile changes from an inscribed circle to a triangular profile. In this embodiment, the thread profile remains the same distance from the centerline of the cannulation 232, creating a tapered thread 224 to a larger body profile. This increases fixation toward end 228 and acts as a pulling feature to the back triangular section near the head 230. The flattened sections 236 may be non-threaded. The implant 240 may include a self-harvesting channel and/or may include optional central lumens or graft windows 238. The triangular outer shape of the sacroiliac joint screw 240 is configured to limit rotational motion and promote fusion.

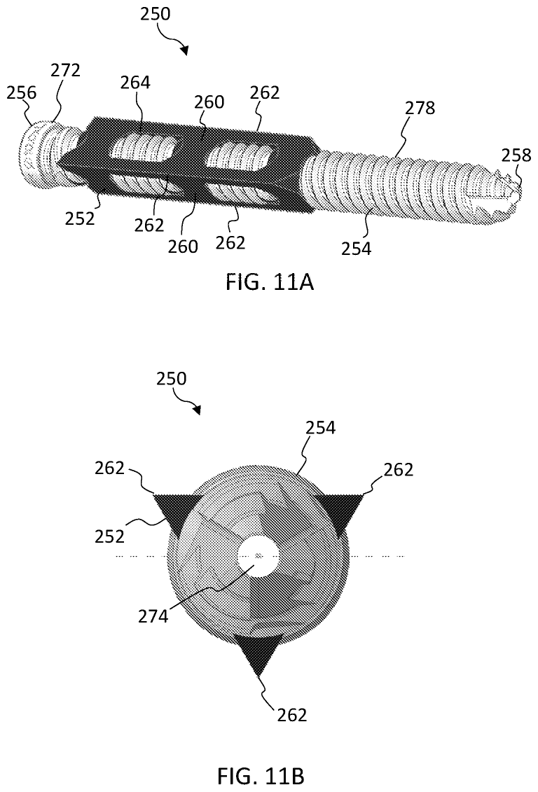

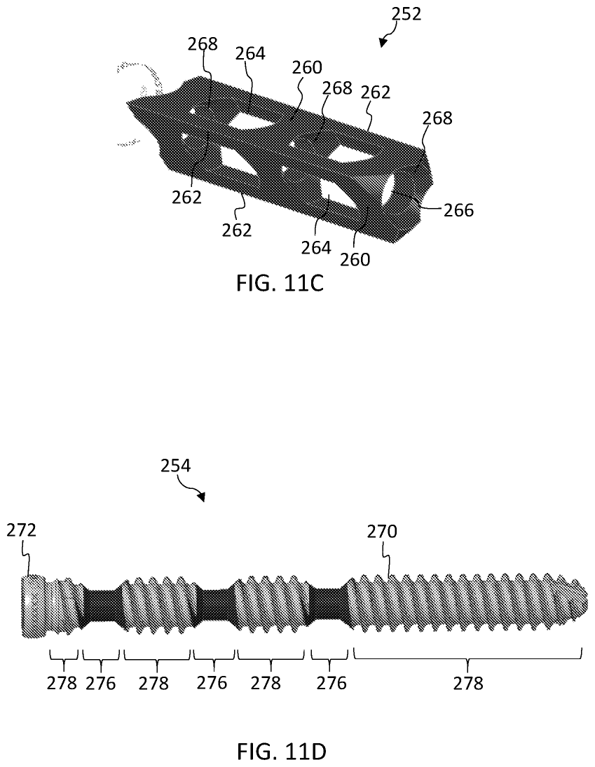

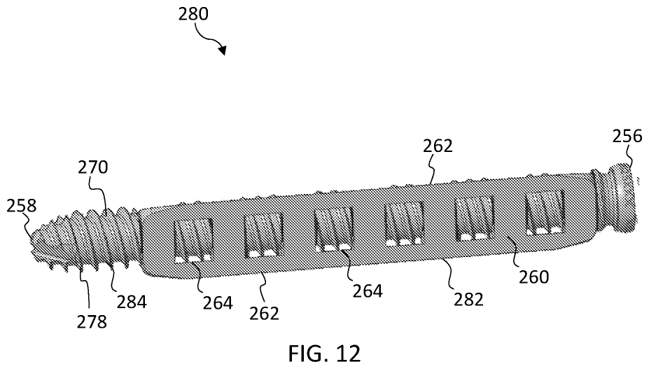

Turning now to FIGS. 11A-11D, an orthopedic fixation device, bone fastener assembly, or triangular implant 250 is shown according to one embodiment. In this embodiment, the implant 250 includes an outer triangular cage 252 encapsulating an inner driving screw 254. The implant 250 extends from a proximal end 256 to a distal end 258 along a central longitudinal axis. The triangular cage 252 is free to rotate relative to the central driving screw 254.

Implant 250 includes outer cage 252, which may have a polygonal outer shape or cross-section, such as a generally triangular cross-section. The structure of the cage 252 may include struts or supports forming the generally triangular shape. As best seen in FIG. 11C, the triangular cage 252 may include three sides or faces 260 and three corners or vertices 262. The sides 260 may be planar or curvilinear forming sharpened corners or vertices 262. In one embodiment, the sides 260 are planar and define one or more openings or windows 264. For example, each side 260 may define a pair of windows 264. The windows 264 may be in fluid communication with a central cavity defined within the structure of the cage 252. The cage 252 may include a bore 266 aligned along the central longitudinal axis configured to receive the body of the screw 254. One or more internal narrowed areas 268 of cage 252 may be configured to mate with corresponding narrowed sections 276 along the screw 254. For example, three narrowed areas 268 within cage 252 may have a reduced diameter along bore 266 sized and dimensioned to receive three respective narrowed sections 276 along screw 254.

The internal screw 254 includes a shaft 270 with an enlarged head 272 at the proximal end. The head 272 may be partially or fully rounded, cylindrical, spherical, or otherwise configured to engage bone. The proximal portion of the head 272 may define a drive recess, such as a hexalobular socket, for example, that can be engaged by a driving instrument. The shaft 170 terminates at the distal end with a distal tip, which may be blunt, pointed, or otherwise configured to engage bone. The shaft 270 may include a central cannulation 274, extending through the entire screw body, along the central longitudinal axis.

As best seen in FIG. 11D, the shaft 254 includes one or more narrowed sections 276 and alternating threaded section 278. The threaded sections 278 may include one or more external threads with a helical ridge defined by a helical groove wrapped around the shaft 270. It will be appreciated that the threads may have a number of different features, such as lead(s), thread pitch, thread angle, shaft diameter to thread diameter, overall shaft shape, and the like configured to engage bone. The narrowed sections 276 have a reduced diameter relative to the threaded sections 278. The lead into the narrowed section 276 from adjacent threaded sections 278 may be angled or sloped to account for the difference in outer diameter. The narrowed sections 276 may be non-threaded and generally cylindrical in cross-section. The narrowed sections 276 are configured to receive the narrowed areas 268 within the triangular cage 252. The narrow channels 276 act as positive stops for the cage 252, preventing the cage 252 from translating along the axis of the screw 254. Although three narrowed sections 276 are exemplified, it will be appreciated that any suitable number of narrowed sections 276 may be selected to retain the cage 252 depending on the type or length of the cage 252 or other factors.

During manufacture, both components 252, 254 of the implant 250 may be 3D printed together. In this manner, the implant 250 has a single body with a breakaway feature. After the implant 250 is printed, the cage 252 breaks from the central screw 254 and is permitted to spin freely about the centerline of the cannulation 274. When implanted into the patient, the cage 252 and screw 254 are not coupled together. The triangle cage 252 is free to rotate, which allows for minimal displacement of the patient tissue. If the triangular cage 252 was coupled to the central screw 254, the profile of the displaced boney material would be a circumscribed cylinder that would encompass the cage 252. By letting the cage 252 freely rotate on the screw 254, the cage 252 is able to pull through the bone in line with the screw insertion axis without rotating about the cannulation axis. While inserting the implant 250, the screw 254 is able to rotate but the cage 252 does not. Once fully seated, the cage 252 acts as a triangular dowel while the screw 254 acts as an assured fixation mechanism.

The implant 250 may include a lag feature by removing or changing the pitch of the threaded portion supporting the cage 252. Lumens or graft windows may be added to any section of the screw 254 if desired. The gaps between the thread and the cage 252 act as lumens to fuse once in its final position in the patient, thereby increasing boney on-growth until fusion occurs. The cage 252 may vary in length and window size depending on length and necessary force to insert the implant 250.

Turning now to FIG. 12, another embodiment of a bone fastener assembly or triangular implant 280, similar to implant 250 and labeled with like reference numbers, is shown. Implant 280 includes an outer triangular cage 282 encapsulating an inner driving screw 284, which are free to rotate with respect to one another. In this embodiment, the cage 282 is longer in length and includes seven narrowed portions 268 configured to engage with seven corresponding narrowed shaft portions 276 defined along the length of the shaft 270. The narrowed portions 276 again act as positive stops for the cage 282 to prevent the cage 282 from translating along the axis of the screw 284. As shown, six windows 264 are provided along each face 260 of the cage 282 between each of the mating areas 268, 276. It will be appreciated that the configuration and length of the cage 252, suitable number of narrowed interfaces 268, 276, and type and number of windows 264 may be selected to optimize fusion and patient outcomes.

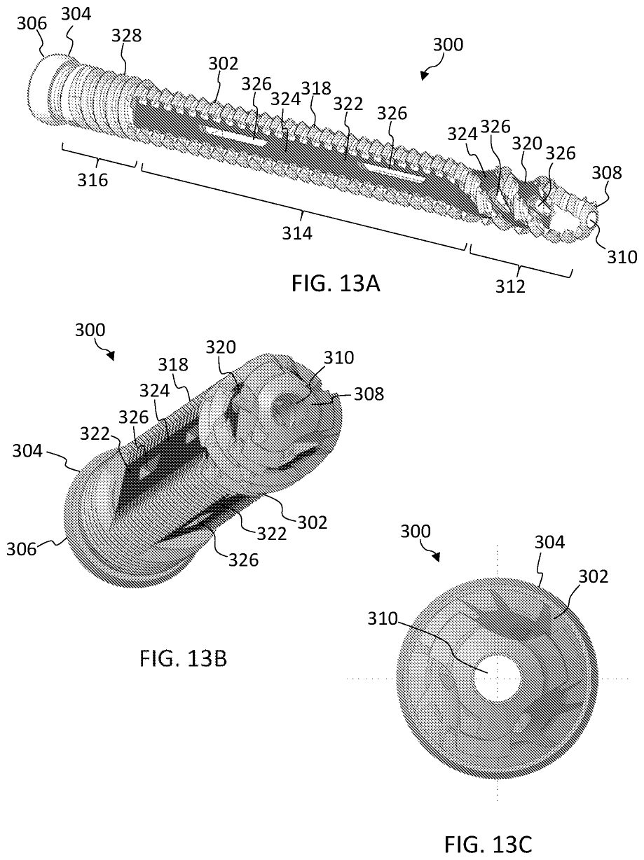

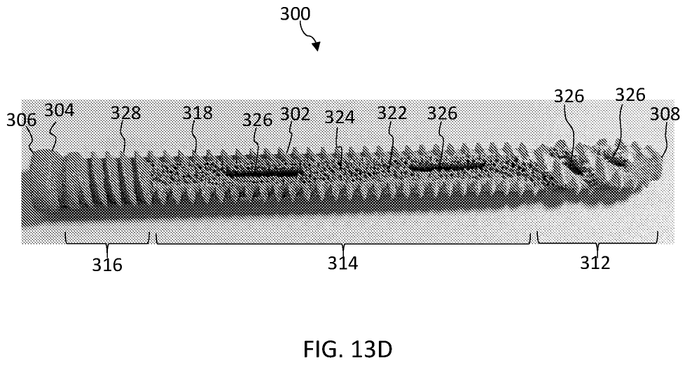

Turning now to FIGS. 13A-13D, an orthopedic fixation device, bone fastener, or triangular implant 300 is shown according to one embodiment. Implant 300 is similar to implant 140 with one or more flat surfaces 322 configured to limit rotational motion and a series of threads or flutes 318 to cut and self-harvest bone to provide faster fusion. The implant 300 may include a shaft 302 extending from a proximal end 306, with an enlarged head 304, to a distal end 308 along a central longitudinal axis. The implant 300 may define a central cannulation 310 therethrough.

In this embodiment, the shaft 302 may include three distinct sections: distal section 312, middle section 314, and proximal section 316. The distal section 312 may include a tapered nose or distal tip and the start or lead in of threads or flutes 318. The profile of the implant 300 may include a circumscribed circle for the first section 312 with spiral flutes 318 and lumens 326 configured to promote fusion through the end 308 of the screw 300 to help with toggle. A helical sweep 320 may be provided as a transition from the middle section 160 to the distal section 312. The helical sweep 320 may transition from the flat surface 322 of middle section 314 into the circular geometry of the distal section 312. The helical sweep 320 may include an area defining a lattice structure 324.

The middle section 314 may include a triangular profile with three planar or flat surfaces 322. The flat surfaces 322 may interrupt the threads or flutes 318 to provide for lattice areas 324. The darkened area represents an area of lattice 324. The lattice structure 324 may form a geometry that includes a plurality of microscopic struts with porosity oriented in a trabecular fashion, for example, to promote bone fusion. Examples of lattice structure are described in U.S. Publication Nos. 2018/0338838 and 2020/0345506, which are incorporated by reference herein in their entireties for all purposes. The boney material may become trapped in the lattice 324 to promote boney on-growth throughout the length of the body. One or more optional openings or graft windows 326 may be provided through the shaft 302. Along the middle section 314, the graft windows 326 may include elongate openings coaxial with the length of the shaft 302. Along the distal section 312, the graft windows 326 may include a triangle, trapezoid, or teardrop shape, for example.

The rear section 316 may include a cylindrical portion of shaft 302 with a helical flute or exterior thread 328. This profile may be configured to move boney material to the area around the middle section 314 of shaft 302. In particular, the helical flutes 328 along section 316 may help to channel the boney material to the triangular cross-sectional central portion 314 of the body. The implant 300 may optionally include lag functionality, such as a washer, or may have a different pitch on the screw thread portion toward the head 304 of the screw 300. The triangular outer shape of the sacroiliac joint screw 300 is configured to limit rotational motion while the flutes 318 are configured to cut and self-harvest bone to provide faster fusion.

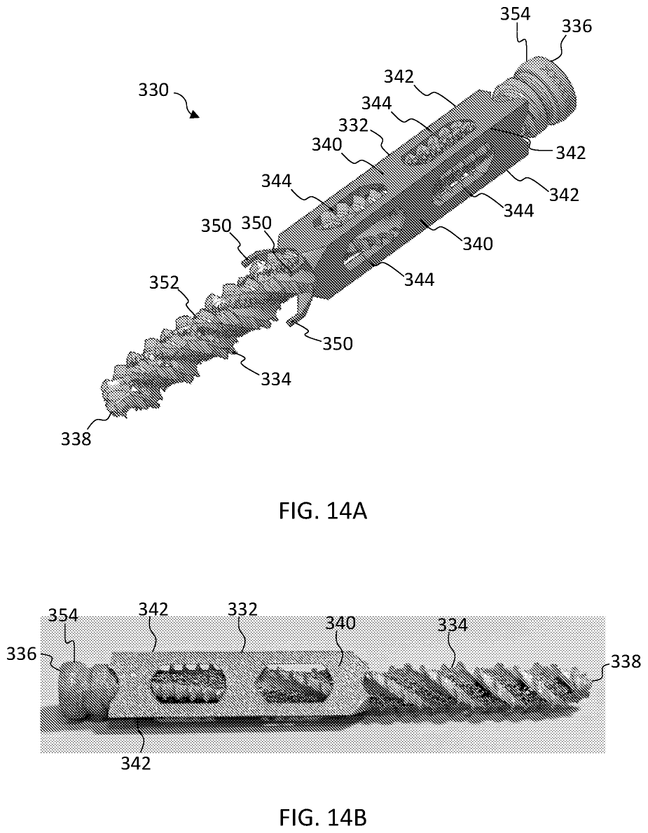

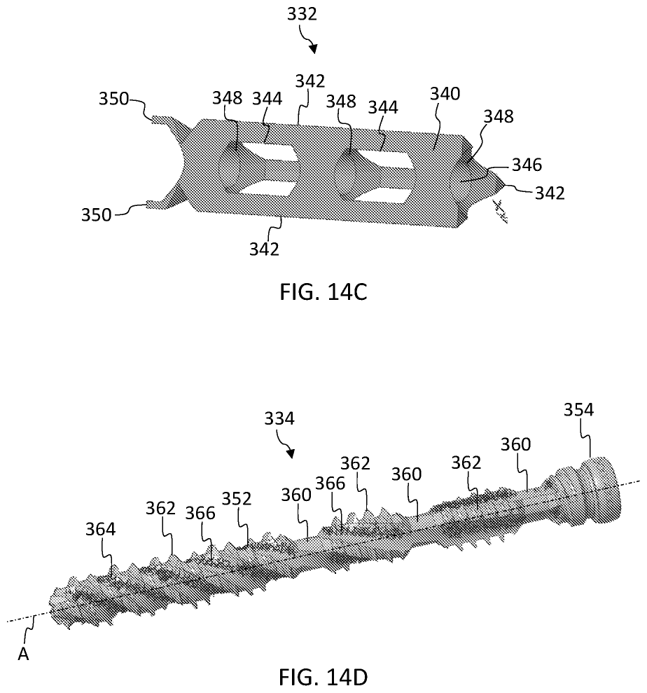

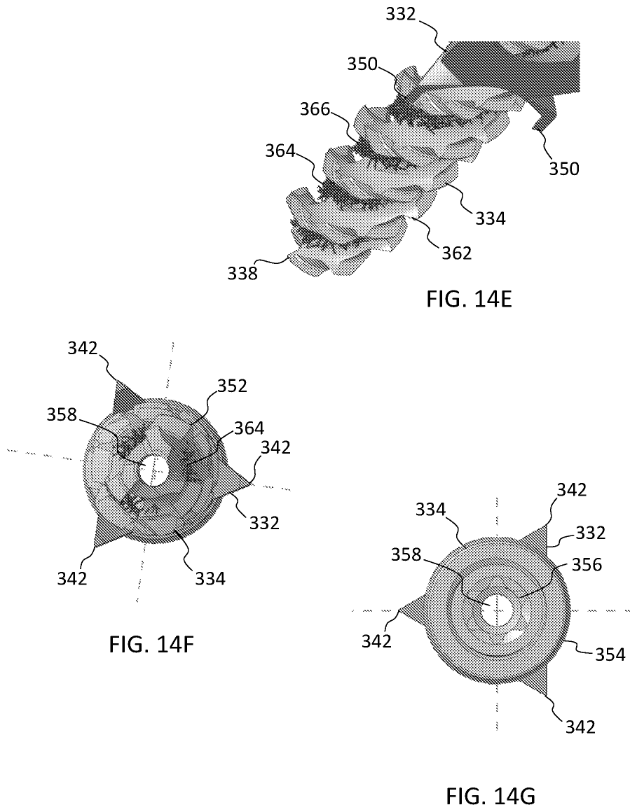

Turning now to FIGS. 14A-14G, an orthopedic fixation device, bone fastener assembly, or triangular implant 330 is shown according to one embodiment. Implant 330 combines elements from implants 250 and 300. The implant 330 includes an outer triangular cage 332 encapsulating an inner driving screw 334. The implant 330 extends from a proximal end 336 to a distal end 338 along a central longitudinal axis A. The triangular cage 332 and central drive screw 334 are free to rotate relative to one another.

Implant 330 includes outer cage 332, similar to cage 282, which may have a polygonal outer shape or cross-section, such as a generally triangular cross-section. As best seen in FIG. 14C, the triangular cage 332 may have a structure with struts or supports defining the body of the cage 332. The cage 332 may include three sides or faces 340, such as planar faces, and three corners or vertices 342, such as sharpened vertices. Each face 340 may define one or more openings or windows 344. The windows 344 may be in fluid communication with a central cavity and a bore 346 configured to receive the body of the screw 334. One or more internal narrowed areas 348 in cage 332 may be configured to mate with corresponding narrowed sections 360 along the screw 334. The cage 332 may include one or more prongs 350 extending distally from the cage 332. For example, three prongs 350 may extend distally from each corner 342 of the cage 32. Each prong 350 may have an L-shaped body with two arm portions including a first arm portion extending laterally and then a second arm portion extending distally. The second distal arm portion may be aligned with each edge of the vertices 342. The free ends of the distal prongs 350 may be configured to engage bone upon insertion and provide additional rotational stability.

With reference to FIG. 14D, the internal screw 334 includes a shaft 352 with an enlarged head 354 at the proximal end 336. As best seen in FIG. 14G, the proximal face of the head 354 may define a drive recess 356, such as a hexalobular socket, for example, that can be engaged by a driving instrument. The shaft 352 terminates at the distal end 338 with a distal tip, which may be blunt, pointed, or otherwise configured to engage bone. The shaft 352 may include a central cannulation 358, extending through the entire screw body, along the central longitudinal axis A.

The shaft 352 includes one or more narrowed sections 360 configured to receive the narrowed areas 348 within the triangular cage 332. The narrowed sections 360 of the shaft 352 have a reduced diameter relative to the remainder of the shaft 352. The lead into the narrowed section 360 may be angled or sloped to account for the difference in outer diameter. The narrowed sections 360 may be non-threaded and non-cylindrical. The cross-section of the narrowed sections 360 may vary or may follow the helical profile or of the shaft 352. The narrow channels 360 act as positive stops for the cage 332, preventing the cage 332 from translating along the axis of the screw 334. Although three narrowed sections 360 are exemplified, it will be appreciated that any suitable number of narrowed sections 360 may be selected to retain the cage 332.

The shaft 352 includes one or more exterior threaded or fluted sections 362. The flutes 362 may each include curvilinear grooves or recesses defining a cutting edge. A helical or spiral sweep 364 may also be provided along the fluted sections 362. The pitch, radius, and or number of turns of the helix 364 may vary along the length of the screw 334. For example, the distal end of the shaft 352 may have a greater number of turns of the helix 364 than the proximal end 336. The pitch of helix 364 may increase from the distal end 338 to the proximal end 336. The spiral sweep 364 may interrupt the flutes 362 to provide for latticed areas 366. The lattice structure 366 may form a geometry that includes a plurality of microscopic struts with porosity oriented, for example, in a trabecular fashion or may be otherwise configured to promote bone fusion. The boney material may become trapped in the lattice 366 to promote boney on-growth throughout the length of the body.

The cage 332 minimizes any additional volume of boney material not being displaced by the volume of the implant 300, while evenly distributing the boney material volume over the entirety of the implant 330 to promote rapid boney on-growth and fusion. The curvature of the cutting edge of the thread profile channels any boney material moved into the lumens but the partially latticed flute surface 366 prevents the boney material from leaving the channel. The triangular outer profile of cage 332 provides the autorotational advantage. While inserting the implant 330, the screw 334 is able to rotate but the cage 332 does not. Once fully seated, the cage 332 acts as a triangular dowel to provide rotational stability while the screw 334 acts as a secure fixation mechanism.

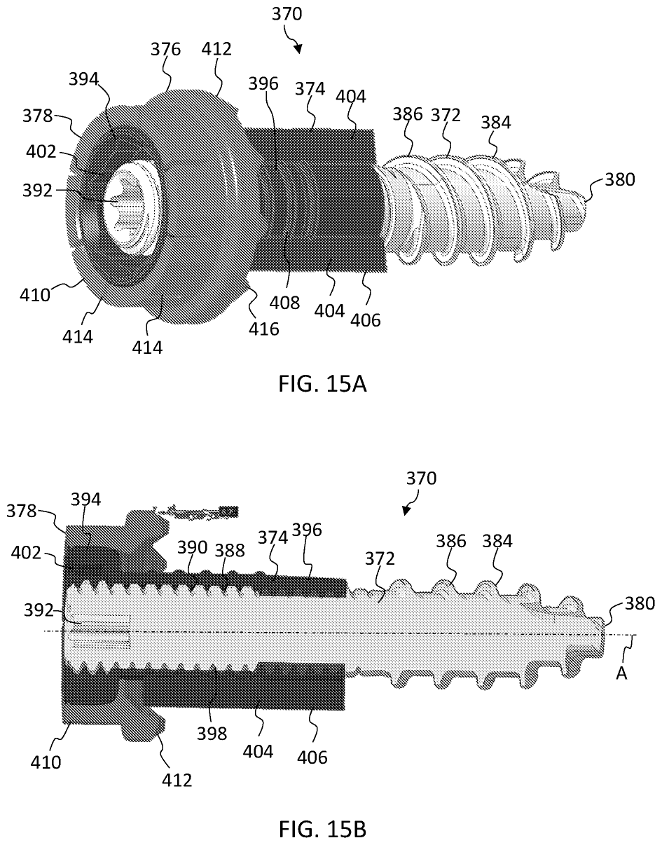

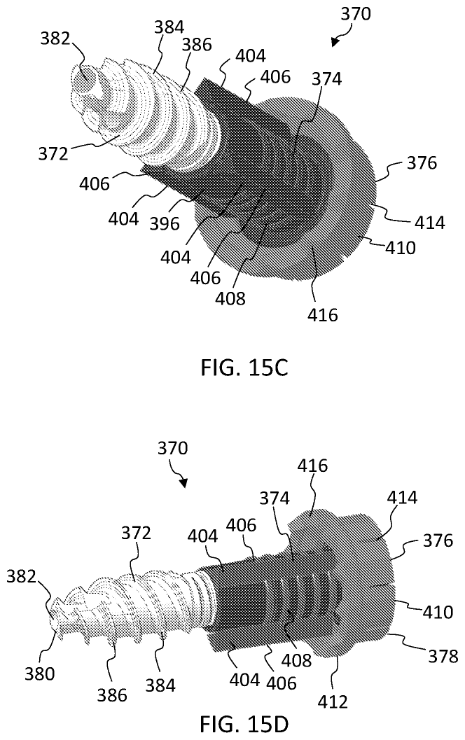

Turning now to FIGS. 15A-15D, an orthopedic fixation device, bone fastener assembly, or triangular implant 370 is shown according to one embodiment. The implant 370 includes a screw shaft 372, a triangular sleeve or cage 374, and a washer 376, configured to provide compression across the sacroiliac joint. The washer 376 creates lag screw functionality and the addition of the cage 374 having a triangular cross-section provides additional rotational stability of the joint as it fuses. The implant 370 extends from a proximal end 378 to a distal end 380 along a central longitudinal axis A. The implant 370 may define a central cannulation 382 therethrough.

As best seen in the cross-sectional view of FIG. 15B, the screw shaft 372 includes a bone engaging portion 384 with one or more external threads 386 configured to engage bone and a cage engaging portion 388 with one or more external threads 390 configured to interface with corresponding internal threads 398 inside cage 374. The bone engaging portion 384 may have a suitable diameter and threads 386 having appropriate lead(s), thread pitch, thread angle, shaft diameter to thread diameter, overall shaft shape, and the like configured to engage bone. The distal tip may be blunt, pointed, or otherwise configured to engage bone. The cage engaging portion 388 may include threads 390 suitable to interface with the sleeve or cage 374. In one embodiment, the threads 386 of the bone engaging portion 384 are a dual lead thread type and the threads 390 of the cage engaging portion 388 are provided with a standard machine thread. It will be appreciate that any type of thread for either portion 384, 388 may be used to facilitate the function of the implant 370. The proximal face of the shaft 372 defines a drive recess 392, such as a hexalobular socket, for receiving a driving instrument.

The sleeve or cage 374 includes a head 394 and a body 396 defining a central through bore with threads 398 configured to mate with the threads 390 on the sleeve engaging portion 388 of the shaft 372. The head 394 of the cage 374 may include a rounded or spherical head having a recess 402 for receiving another driving instrument. As best seen in FIG. 15C, the body 396 of the cage 374 may include one or more bars 404 extending longitudinally along the length of cage 374. Each bar 404 may have a triangular shape with smooth planar faces and a sharpened corner or vertex 406, which projects radially outward. The arrangement of three triangular bars 404, for example, equally spaced around the periphery of the cylindrical body 396 provides a generally triangular cross-section for cage 374. It will be appreciated that the number and configuration of bars 404 may be varied. The outer cylindrical surface of body 396 may include one or more threads 408 interrupted by each of the triangular bars 404. The threads 408 may have a suitable angle, lead, pitch, etc. to enhance engagement with the bone. The cage 374 is configured to freely rotate around the central axis A of the screw 372.

The washer 376 may include a proximal flexible portion 410 and a distal bone engaging portion 412. The washer 376 defines an opening having a rounded or spherical curvature for retaining the head 394 of the cage 374. The flexible portion 410 may define a plurality of slits 414 configured to allow the washer 376 to be flexible or resilient for engaging with the head 394 of the cage 374. The bone engaging portion 412 is configured and dimensioned with a plurality of protrusions 416 to engage with bone. The washer 376 is configured to angulate with respect to the cage 374 and screw shaft 372, thereby enabling the washer 376 to conform to various angles of the bone while the implant 370 is advanced across the sacroiliac joint.

The implant 370, including the screw shaft 372 and the cage 374, may have added lumens or graft windows. The components may be 3D printed in three pieces or may be machined and assembled together. When the outer cage 374 is rotated, the cage 374 advances distally along screw shaft 372. As the washer 376 is advanced distally to contact the bone, compression of the bone portions occurs providing greater stability and fixation of joint. Thus, the implant 370 provides compression across the sacroiliac joint and provides additional rotational stability to the joint.

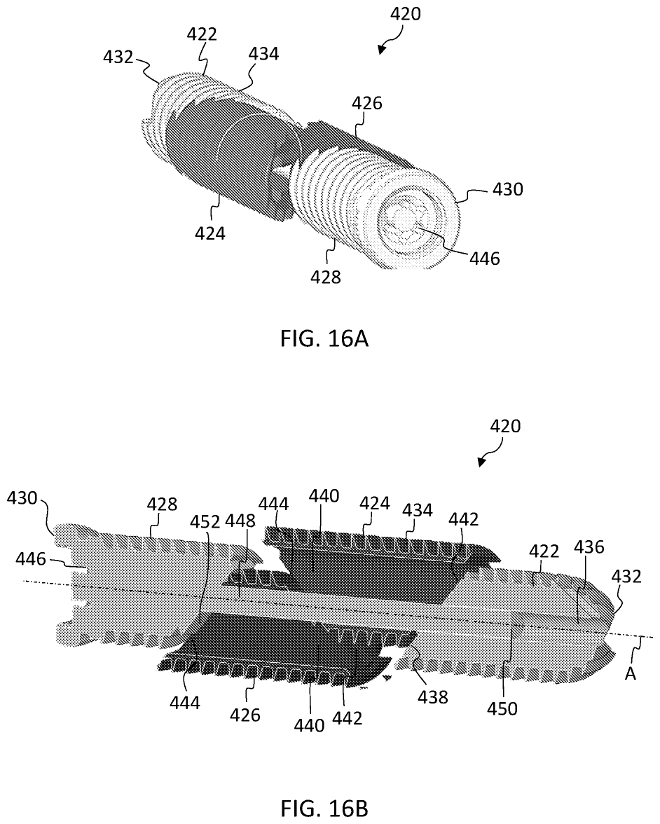

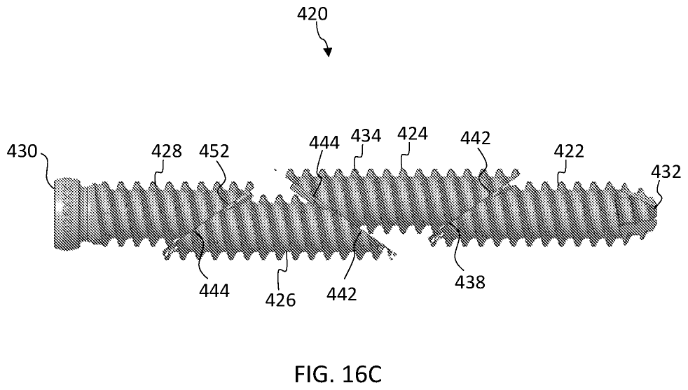

Turning now to FIGS. 16A-16C, an orthopedic fixation device, bone fastener assembly, or expandable implant 420 is shown according to one embodiment. The expandable implant 420 includes a front section 422, a first expandable section 424, a second expandable section 426, and a rear section 428. The implant 420 extends from a proximal end 430 to a distal end 432 along a central longitudinal axis A. The expandable sections 424, 426 are configured to expand away from the central longitudinal axis A. The expandable screw 420 provides compression with additional radial pressure on the bone by translating the compressive force radially in this manner. The implant 420 is configured to apply pressure on the cortical walls of the joint, thereby providing additional fixation.

When collapsed and aligned together, each of the sections 422, 424, 426, 428 of the expandable screw 420 define a shaft with one or more threads 434. The screw thread 44 may include an external thread with a helical ridge defined by a helical groove wrapped around the shaft. The threads 434 may have any suitable angle, lead, pitch, etc. to enhance insertion and/or engagement with the bone.

The front section 422 includes a nose or distal end configured to enter the bone first. The nose may be blunt, pointed, or otherwise configured to engage bone. The front section 422 defines a central through bore 436 aligned along central longitudinal axis A. The bore 426 is configured to receive the internal shaft 448 of the rear section 428 therein. Bore 436 may be internally threaded to threadedly engage with internal shaft 448. The proximal face of the front section 422 includes one or more ramps 438 configured to interface with corresponding mating ramps 442 on the distal side of the first expandable section 424.

The expandable implant 420 includes a pair of expandable central sections 424, 426. Although two expandable sections are exemplified, it will be appreciated that a single central section may be used or more expandable sections may be added to the implant 420. Expandable sections 424, 426 may be identical except for their orientation about axis A. Expandable sections 424, 426 each include a through channel 440 for receiving a portion of the internal shaft 448. The distal face of each expandable section 424, 426 includes one or more ramps 442 configured to interface with the adjacent mating ramps 438, 444 and the proximal face of each expandable section 424, 426 includes one or more ramps 444 configured to interface with the adjacent mating ramps 442, 452. For example, front-facing ramp 442 of the first expandable section 424 slidably mates with rear-facing ramp 438 of the front section 422, rear-facing ramp 444 of the first expandable section 424 slidably mates with front-facing ramp 442 of the second expandable section 426, and rear-facing ramp 444 of the second expandable section 426 slidable mates with front-facing ramp 452 of the rear section 428.

Rear section 428 may include a cylindrical body with an enlarged head having a drive recess 446 at the proximal end 430. Inner shaft 448 extends distally from the body of the rear section 428 and terminates at free end 450. Inner shaft 448 is externally threaded to threadedly connect with the internally threaded bore 436 of the front section 422. The available radial force of implant 420 may be limited by the tensile strength of the internal threaded shaft 448. Rear section 428 includes one or more ramps 452 configured to interface with the adjacent mating ramp 444 of the second expandable section 426. The mating ramps 438, 442, 444, 452 may include angled, sloped, or ramped surfaces, sliding components, male/female members, tongue and groove, dovetail, or other suitable mating interfaces configured to expand sections 424, 426. In one embodiment, ramps 438, 442, 444, 452 include angled continuous surfaces with a given angle of slope configured to complement one another. The ramped surfaces may be sloped upwardly, downwardly, etc. or in any suitable mating fashion to achieve the desired expansion.

Implant 420 may include an optional washer similar to implant 370 or may include lag screw functionality to add extra compression before expansion. The different sections 422, 424, 426, 428 of the expandable screw 420 may be 3D printed to promote boney on-growth. The central threaded rod 448 may also be 3D printed to be under tension while dynamic forces are transferred to the opposite expanded sections 424, 426 of the outer body.

The central expandable sections 424, 426 of screw 420 are configured to expand radially outward. When the rear section 428 is rotated, inner threaded shaft 448 rotates and translates front section 422 toward the rear section 428. As the distance between the front and rear sections 422, 428 is reduced, the force causes the expandable sections 424, 426 to translate along ramps 442, 444, thereby expanding sections 422, 426 outward. The expandable sections 424, 426 are expanded away from the central longitudinal axis A. The expansion of screw 420 provides a compressive radial force on the bone, thereby providing additional fixation of implant 420.

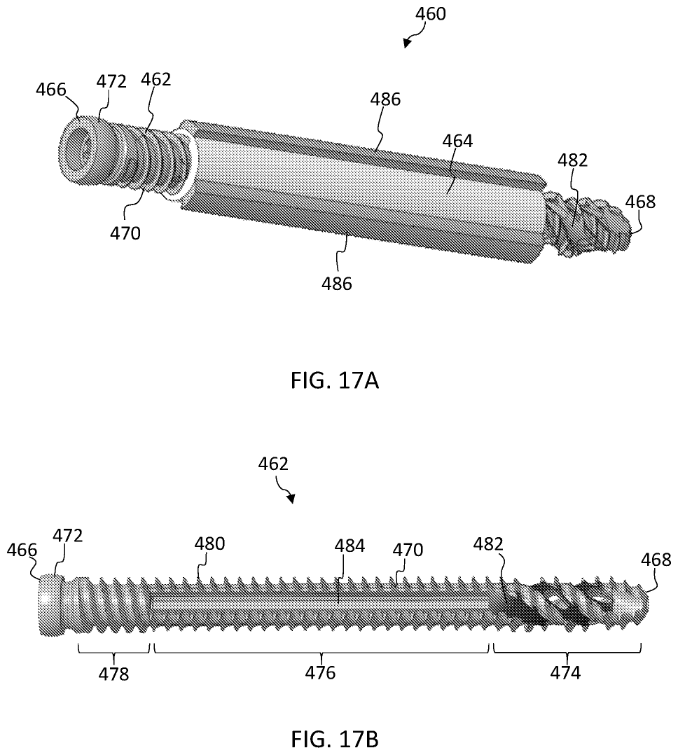

Turning now to FIGS. 17A-17B, an orthopedic fixation device, bone fastener assembly, or expandable implant 460 is shown according to one embodiment. In this embodiment, implant 460 includes a central screw 462 and an expandable outer sleeve 464, which may have a generally triangular cross-section. The sleeve 464 may be configured to radially expand via cement or cement filled balloons, for example. The implant 460 extends from a proximal end 466 to a distal end 468 along a central longitudinal axis.

The central screw 462 includes many of the same features as screw 300. The screw 462 may include a shaft 470 having an enlarged head 472 at the proximal end 466 and a central cannulation therethrough. Similar to screw 300, the shaft 470 may include three distinct sections: distal section 474, middle section 476, and proximal section 478. The distal section 474 may include a tapered nose or distal tip and the start or lead in of threads or flutes 480. A helical sweep 482 may be provided as a transition from the middle section 476 to the distal section 474. The helical sweep 482 may interrupt the threads or flutes 480 and define a smooth surface or optional lattice structure therein. The middle section 476 may define one or more elongated longitudinal windows 484 through the shaft 470. The elongate windows 484 are in fluid communication with the central cannulation. When cement or other suitable material is passed through the screw 462, the cement flows through the central cannulation and out graft windows 484 to expand outer sleeve 464. The rear section 478 may include a cylindrical portion of shaft 470 with a helical flute or exterior thread.

The outer sleeve 464 may be a cylindrical tube having one or more expandable bars 486. The expandable bars 486 may be elongate members extending between the proximal and distal ends of the outer sleeve 464. In one embodiment, three bars 486 may be arranged equidistantly about the sleeve 464 to provide a generally triangular cross-sectional shape. The bars 486 may have smooth outer surfaces, approximating a truncated triangle, or may have an apex or vertex (not shown). The triangular profiled sleeve 464 may be configured to expand, for example, via cement or cement filled balloons. Each expandable bar 486 may align with the respective elongate windows 484 in shaft 470. In this manner, as the cement, other fluid, or balloon flows or emerges through windows 484, the force causes the expandable bars 484 to translate radially outward from the center axis of the implant 460. The expansion of bars 486 provides a compressive radial force on the bone, thereby providing additional fixation of implant 460.

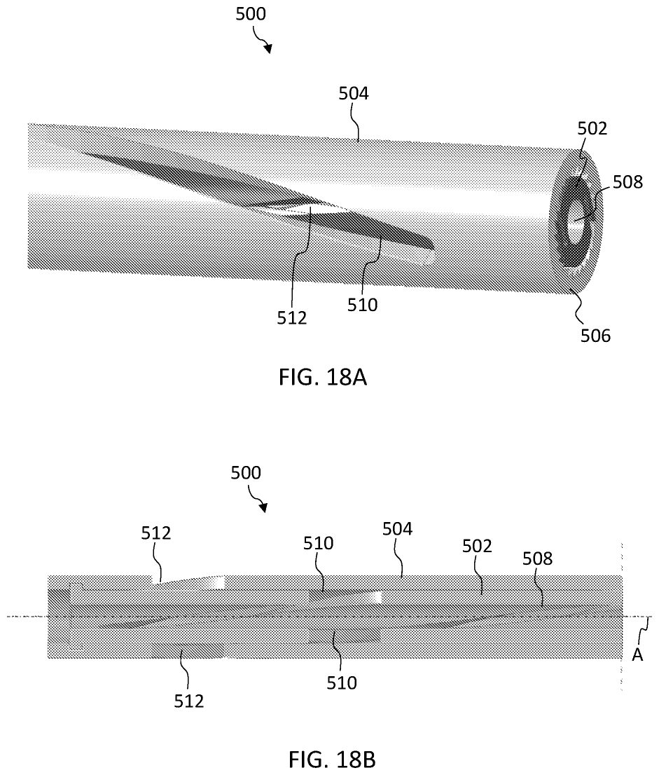

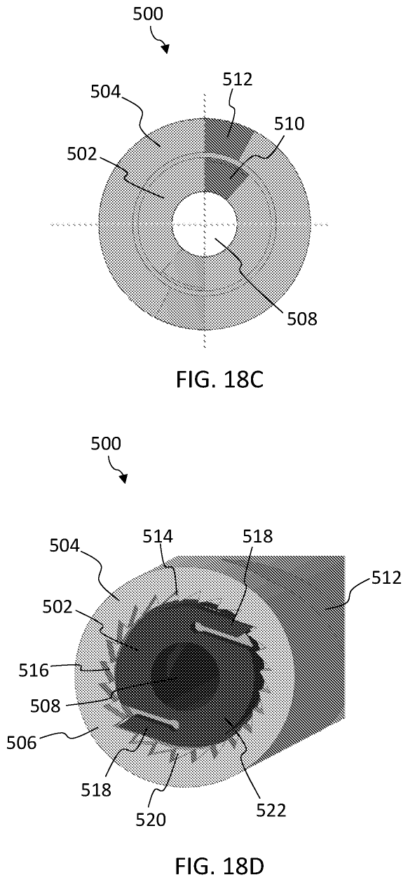

Turning now to FIGS. 18A-18D, an orthopedic fixation device, bone fastener assembly, or implant 500 is shown according to one embodiment. In this embodiment, the implant 500 includes an inner shaft 502 receivable through an outer shaft 504. Rotational movement of inner and outer shafts 502, 504 relative to one another create a movable lumen across the body of the implant 500. The implant 500 extends from a proximal end to a distal end 506 along a central longitudinal axis A.

The inner shaft 502 includes a cylindrical tube with a central cannulation 508 along central longitudinal axis A. The inner shaft 502 defines one or more cuts 510 through the wall of the shaft 502. The cuts 510 may include a helical cut or spiral cut extending along the length of the shaft 502. The outer shaft 504 includes a cylindrical tube sized and configured to receive the inner shaft 502 therein. The outer shaft 504 defines one or more cuts 512 through the wall of the shaft 504. The cuts 512 may include a helical cut or spiral cut extending along the length of the shaft 504. The pitch, radius, and or number of turns of the helical cuts 510, 512 may be different or offset for the two shafts 502, 504. The inner and outer shafts 502, 504 may have helical profile cuts 502, 504 along the central axis A of both inner and outer bodies.

The two helical profiles may achieve a lumen when the cuts 510, 512 of the inner and outer shafts 502, 504 align. As best seen in the axial view of FIG. 18C, as the inner and/or outer shafts 502, 504 are rotated with respect to one another, the cuts 510, 512 may overlap at certain positions. In this manner, the relative position between the two components 502, 504 creates a lumen by having the same pitch of the profile and pitch along the length but extending along the length of the implant 500 in opposite directions. The implant 500 may be inserted to its final position and then the internal shaft 502 is able to be rotated independently allowing interoperative movement of the lumen position. The final positioning may be verified with fluoroscopy.

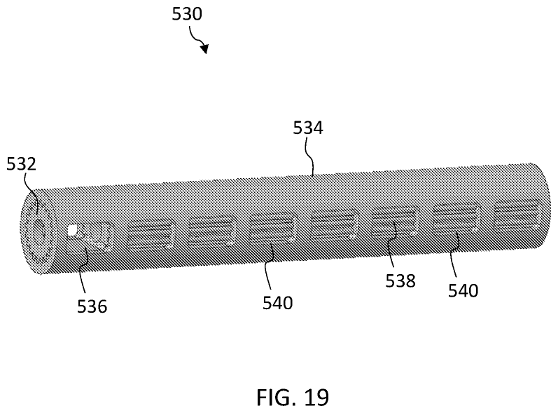

In an alternative embodiment, as shown in FIG. 18D, the lumen placement may be planned and preset pre-operatively by utilizing a one-way lock 514. The one-way lock 514 may include a ratchet 516 and flexing wings 518 configured to provide a one-way stop. The ratchet 516 may include a plurality of teeth 520 defined into the outer shaft 502. The plurality of teeth 520 may be arranged in a circular pattern in the distal end 506 of the outer shaft 502. The teeth 520 may be uniform in shape but asymmetrical to allow for rotational movement in one direction and preventing movement in the opposite direction. The lock 514 includes an end cap 522 attached to or integral with the inner shaft 502. The end cap 522 includes one or more flexing wings 518, which are receivable in respective teeth 520. The flexing wing 518 may be formed by a spring cut forming a free end configured to engage one of the teeth 522. The flexing wings 518 may include a pair of opposite wings pointing in opposite directions. As one of the shafts 502, 504 rotates relative to the other shaft 502, 504, the lock 514 allows continuous rotary motion in only one direction while preventing motion in the opposite direction. In yet another embodiment, the inner and outer shafts 502, 504 may be locked with respect to one another utilizing splines, for example, similar to the embodiment shown in FIG. 19. It will be appreciated that any suitable locking mechanism may be selected to provide the desired arrangement and final positioning between the inner and outer shafts 502, 504.

Turning now to FIG. 19, an orthopedic fixation device, bone fastener assembly, or implant 530 is shown according to one embodiment. Similar to implant 500, implant 530 includes an inner shaft 532 receivable through an outer shaft 534. In this embodiment, the rotatable inner shaft 532 is splined to the outer shaft 534. Implant 530 includes one or more moveable graft windows 536. The inner shaft 532 may include several lumens along its length timed to the splines 538. The outer shaft 534 include a plurality of windows 540 defined through the wall of the shaft 534. For example, a series of rectangular windows 540 may be aligned along the length of the shaft 534. When the inner shaft 532 is rotated relative to the outer shaft 534, the preceding lumen on the inner shaft 532 is configured to align with the preceding lumen of the outer shaft 534. The implant 530 positioning may be pre-operatively planned. The implant 530 may be fitted to the patient anatomy and match the trajectory and approach as determined by the physician. This provides for maximum versatility to match the needs of the patient.

In another embodiment, the implant may include a movable graft window with a modular screw shank. By providing a modular system, different graft window placement may be achieved that locks into the distal tip and/or head of the screw. This may provide ideal placement of the graft widow across the joint to promote fusion. In yet another embodiment, the moveable graft window may be provided in a singular implant with multiple lumens across the length of the implant. The set may provide a variety of lumen inserts to fill the lumens based on doctor preference. The inserts may include biologics, 3D lattice, solid machined inserts, etc.

According to another embodiment, a method for stabilizing a sacroiliac joint may include (a) providing one or more implants of types described herein; (b) accessing an ilium and/or a sacrum of a patient through a lateral approach or a posterior approach (e.g., lateral to medial or medial to lateral); and (c) inserting the implant across the sacroiliac joint, thereby providing fixation and promoting fusion of the two bones. Multiple implants may be inserted across the joint to better stabilize and prevent movement of the sacroiliac joint. The anatomy of the patient may be accessed using a standard or minimally invasive surgical (MIS) technique. The surgery may be performed with the assistance of robotic and/or navigational systems.

Some of the implants described herein may combine the clinical benefit of limiting the rotational range of motion, while being able to provide fixation and precise insertion methods of the implant to provide fusion. Some embodiments provide a device that alters its shape or allows for or promotes boney ingrowth in a way designed to improve bone interface strength compared to insertion geometry. These features may include altered surface texture, 3D printed porous structure, shape changing thread profiles, or expandable implant geometry. Further, a surface treatment may be used to increase the frictional force between internal components in a multitude of areas when locked. In other embodiments, the use of a movable graft window may be incorporated to maximize fusion of the bone based on the anatomy of the patient. The implants may help to minimize the displacement of boney material around the implant. When less material is displaced, better fixation of the implant may result, thereby preventing movement of the joint and allowing fusion to occur. The placement of lumens or windows may help to allow boney on-growth and through growth, for example, to prevent toggle.

In some embodiments, interchangeable components and/or instrumentation may be provided. This may help to reduce the number of sets required in the operating room and to streamline the technique. Using instrumentation across platforms further reduces the manufacturing burden by reducing the number of new instruments required.

It will be further understood that various changes in the details, materials, and arrangements of the parts which have been described and illustrated in order to explain the nature of this invention may be made by those skilled in the art without departing from the scope of the invention as expressed in the claims. One skilled in the art will appreciate that the embodiments discussed above are non-limiting. It will also be appreciated that one or more features of one embodiment may be partially or fully incorporated into one or more other embodiments described herein.

What is claimed is:

1. A method for stabilizing a sacroiliac joint, the method comprising:
providing an implant having an outer cage with a polygonal cross-section and an internal screw aligned along a central longitudinal axis, wherein the outer cage is free to rotate about the central longitudinal axis relative to the internal screw to allow for minimal displacement of patient tissue;
accessing an ilium of a patient; and
inserting the implant across the sacroiliac joint such that once the implant is fully seated, the outer cage acts as a stop to prevent rotational motion of the implant.

2. The method of claim 1, further comprising inserting multiple implants across the sacroiliac joint to better stabilize and prevent movement of the joint.

3. The method of claim 1, further comprising accessing the sacroiliac joint with a robotic and navigational system.

4. A method for stabilizing a sacroiliac joint, the method comprising:
providing an implant comprising:
a shaft extending along a central longitudinal axis having a distal tip configured to facilitate insertion into bone and a proximal end configured to be engaged by an instrument, the shaft defines an external thread configured to enhance insertion and/or engagement with bone,
wherein a portion of the shaft includes a polygonal portion with a triangular cross-section having three faces, and wherein the polygonal portion is configured to limit rotational motion about the central longitudinal axis of the shaft,
positioning the implant into the sacroiliac joint.

5. The method of claim 4, wherein the polygonal portion is an equilateral triangle with three equal faces.

6. The method of claim 4, wherein the three faces define three vertices at each corner.

7. The method of claim 1, wherein the three faces are planar.

8. The method of claim 4, wherein the polygonal portion is a core of the shaft.

9. The method of claim 4, wherein the polygonal portion defines a lattice structure.

10. The method of claim 4, wherein the thread includes a helical thread.

11. The method of claim 4, wherein a graft window extends longitudinally through the shaft.

12. A method for stabilizing a sacroiliac joint, the method comprising:
providing an implant comprising:
an outer cage and an internal screw aligned along a central longitudinal axis, the outer cage having a polygonal outer shape configured to limit rotational motion, and
the internal screw is sized and dimensioned to fit through the outer cage, the internal screw having an enlarged head and a shaft with one or more threaded sections, wherein the outer cage is free to rotate relative to the internal screw, positioning the implant in the sacroiliac joint.

13. The method of claim 12, wherein the outer cage has a triangular cross-section.

14. The method of claim 12, wherein the outer cage include three faces and three vertices.

15. The method of claim 12, wherein the shaft of the internal screw includes one or more narrowed shaft portions, and the outer cage defines corresponding narrowed sections configured to mate with the narrowed shaft portions, wherein the narrowed shaft portions act as stops to prevent the outer cage from translating along the central longitudinal axis.

16. The method of claim 15, wherein the narrowed shaft portions are non-threaded and have a reduced diameter relative to the threaded sections.

17. The method of claim 2, wherein the shaft includes three distinct sections including a distal section with a tapered nose, a middle section having a triangular profile, and a rear section having a cylindrical profile.

18. The method of claim 12, wherein the outer cage includes one or more prongs extending distally from the outer cage.

19. The method of claim 12, wherein the implant includes a spiral sweep interrupting the threaded sections.

20. The method of claim 16, wherein the spiral sweep includes a lattice structure.
